(19) United States
(12) Patent Application Publication
(10) Pub. No.: US 2003/0216689 A1
(43) Pub. Date: Nov. 20, 2003

(54) Apparatus for heating blood or another physiological fluid

(76) Inventors: Menno Cornelis Bouhuijs, Hengelo (NL); Petrus Theodous Rutgers, Hengelo (NL)

Correspondence Address: PEACOCK MYERS AND ADAMS P C, P O BOX 26927, ALBUQUERQUE, NM, 871256927

(21) Appl. No.: 10/371,684

(22) Filed: Feb. 20, 2003

Related U.S. Application Data
(63) Continuation of application No. PCT/NL01/00596, filed on Aug. 10, 2001.

(30) Foreign Application Priority Data
Aug. 23, 2000 (NL) NL 1015999

(51) Int. Cl. A61F 7/12; H05B 1/00

(52) U.S. Cl. 604/113

(57) ABSTRACT

The invention relates to an apparatus for warming blood or other physiological fluids, comprising a conduit through which the fluid can be conducted, and heating means for warming the fluid flowing through the conduit, the heating means comprising at least one source of infrared radiation, and the conduit being at least partly transparent to the radiation emitted by the infrared source during operation.

The invention relates to an apparatus for warming blood or other physiological fluids, comprising a conduit through which the fluid can be conducted, and heating means for warming the fluid flowing through the conduit.

Such an apparatus is known from the international patent application PCT/US96/04737 (WO 96/32080). The apparatus disclosed therein serves for warming blood and has a transparent conduit so that the blood flowing through the conduit is visible. The blood that flows through the conduit is warmed due to the fact that the conduit is fabricated from an electrically conductive polymer material, so that resistance heating of the material, and thus indirectly of the blood, can take place. A drawback of this known apparatus is that the heating is relatively inefficient, that there is a rather long response time, and is consequently difficult to control.

One object of the invention is to realize an improvement with respect to these points, and to realize a more direct warming of the blood or of the physiological fluid to be warmed in general, such as to provide a better controllability of the fluid temperature. A further object of the invention is to provide an apparatus with which the blood or other physiological fluid can the processed hygienically, and with which the development of so-called hot spots in the (blood) stream is avoided, as occurs with direct heating by means of microwave techniques such as, for example, disclosed in the American patent U.S. Pat. No. 5,073,167.

A further object of the invention is to avoid problems inherent to heating by applying electrical energy, in particular the risk of current leaking to a patient receiving the warmed blood or other physiological fluid from the apparatus.

From EP-A-0 138 489 an apparatus is known for the irradiation of somatic cells, in particular the treatment of blood cells, with ultraviolet radiation. To this end a tubular construction is used wherein the radiation source is arranged coaxially. The blood fluid is conducted through the tube wherein the fluid is bordered on one side by the radiation source and on the other side by the wall of the tube in which the radiation source is placed.

WO-A-97/46271 discloses an apparatus for warming blood or other physiological fluids, comprising a conduit through which the fluid can be conducted, and heating means for warming the fluid flowing through the conduit, wherein the heating means comprise at least one source of infrared radiation, the conduit is at least partly transparent too the radiation emitted by the infrared source during operation, and wherein the conduit, at its discharge, is provided with a temperature sensor that is coupled to, and feeds a control member.

In this apparatus a separately adjustable unit is used for controlling the temperature of the fluid that is conducted through the conduit.

The apparatus according to the invention is characterized in that the infrared source be adjustable and connected to the control member. Surprisingly, such an infrared source provides a very well controllable, direct warming of the blood or the fluid flowing through the transparent conduit. Because there is no direct contact at all, such heating can take place very hygienically, and because there is no electrical contact with the conduit or the fluid flowing through the conduit, the risk of leakage currents is totally absent.

The fluid flowing through the conduit may be warmed very efficiently in a first preferred embodiment of the apparatus, which is characterized in that the conduit defines a space wherein the infrared source is placed, and in that the conduit, at least at the side facing the infrared source, is transparent to infrared radiation.

A very suitable second embodiment of the apparatus is characterized in that the conduit extends substantially in a plane, in that the infrared source is situated next to the conduit, and in that the conduit, at least at the side of the source, is transparent to infrared radiation. This allows the blood or the fluid to be warmed safely and simply.

The efficiency and controllability is aided in particular by the fact that the conduit extends substantially in the form of a spiral and that its side facing away from the infrared source is provided with an infrared radiation-reflective coating or film. Such an embodiment of the apparatus according to the invention can, moreover, be produced at relatively low costs.

It is further advantageous for the apparatus to be equipped with a flowmeter connected with the control member, in order to be able to accurately adjust the widely varying currents of blood or other physiological fluid that flows through the apparatus.

A first embodiment with which this may be realized is characterized in that the flowmeter comprises a heat source, and at least one temperatures sensor.

A second preferred embodiment is characterized in that the flowmeter comprises two temperature sensors and a heat source positioned between said temperature sensors or near the temperature sensors positioned at the discharge side.

The invention will now be further elucidated with reference to the drawings, which

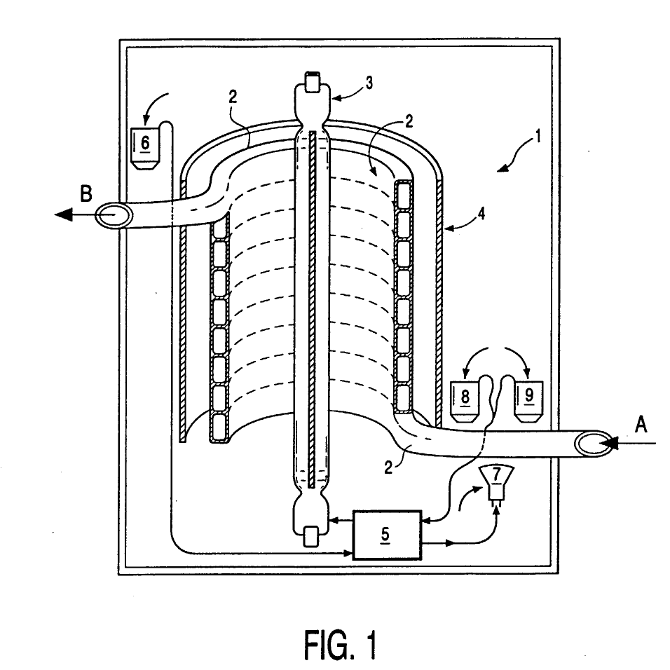

in FIG. 1, schematically show a longitudinal section of a first preferred embodiment of the apparatus according to the invention; and

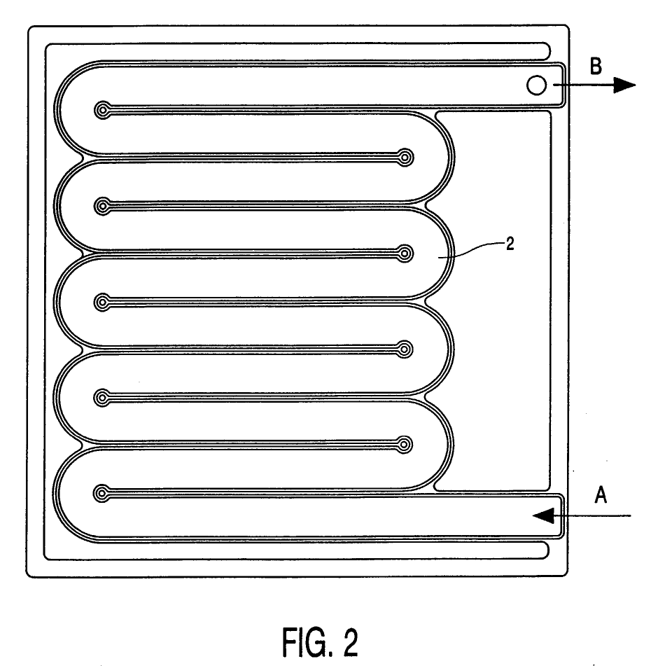

in FIG. 2, show a top view of the conduit that may be used in a second preferred embodiment of the apparatus according to the invention.

The general workings of the apparatus according o the invention will now first be elucidated with reference to FIG. 1.

The apparatus 1 according to the invention for arming blood or other physiological fluids comprises a conduit 2, wherein the entrance is indicated by arrow A and the exit by arrow B. Through this conduit 2 the blood or the physiological fluid to be warmed is conducted by means of propulsion, which in itself is known to a person skilled in the art, and which requires no further elucidation at this point. The Figure shows that the conduit 2 is formed like a spiral, and that in the interior of the spiral shape an infrared source is provided, for example, an infrared lamp 3, which serves to warm the blood or another physiological fluid flowing through the conduit 2. For this purpose the conduit 2 is transparent to infrared radiation, at least at the side facing the infrared lamp 3. It is preferred, however, for the side of the conduit 2 facing away from the infrared lamp 3 to be transparent also, and for this latter side to be provided with a heat-reflective coating or film 4. The infrared lamp 3 is preferably adjustable, being connected to a control member 5. Also connected with the control member 5 is a temperature sensor 6, positioned at the discharge side of the conduit 2. In this way the power supply of the adjustable infrared lamp 3 is rendered dependent on the temperature values of the blood or another physiological fluid that leaves the conduit at the discharge side near arrow B. Conveniently there is also a flowmeter 7, 8, 9 connected to the control member 5, to allow the control member 5 to adjust the power supply of the infrared lamp 3 subject to the amount of blood or other physiological fluid being conducted through the conduit 2. A possible embodiment of the flowmeter 7, 8, 9 is one in which a heat source 7 is used, together with one single temperature sensor 8. The flow rate of the blood or fluid can be determined by, with the aid of the heat source 7, supplying the blood or the other physiological fluid entering the conduit 2 near arrow A with heat pulses at a predetermined known frequency, and by measuring these with the aid of the temperature sensor. The Figure shows another embodiment wherein the flowmeter 7, 8, 9 comprises two temperature sensors 8 and 9, and wherein the heat source 7 is positioned between the temperature sensors 8 and 9. The measured temperature difference between the temperature sensors 8 and 9, together with the amount of heat introduced by the heat source 7, determine the flow rate of the blood or the fluid entering the conduit 2 near the arrow A. It should be noted that the heat source 7 may also be positioned near (opposite to) the temperature sensor 8 near the discharge side.

The foregoing description is to be understood as being a non-limiting exemplary embodiment of the apparatus according to the invention without limiting the protection merited by the appended claims. The example given merely serves to elucidate said claims.

It is, for example, also possible not to embody the conduit 2 as a cylindrical spiral defining a space for housing the infrared lamp 3 but to embody the spiral of the conduit 2 in a plane. A top view of this spiral form of the conduit is shown in FIG. 2. Blood or another physiological fluid to be warmed is introduced into the conduit 2 at the entry indicated with arrow A, and leaves the conduit 2 at the exit marked with arrow B. In this embodiment the infrared lamp or lamps are positioned next to the conduit 2 in a manner that is completely obvious to the person skilled in the art, and requires no further elucidation.

In correspondence to the afore-described first preferred embodiment, the conduit 2 is, at least at the side of the lamp 3 or lamps, transparent to infrared radiation, and at the side facing away from the infrared lamp or lamps a heat-reflective coating or film may be provided. The infrared lamp or lamps are adjusted in accordance with the manner described for the first preferred embodiment shown in FIG. 1. In practice, said second preferred embodiment, in which the conduit 2 is arranged in a plane is for production technical reasons preferred. The blood or the other physiological fluid conducted through said conduit can be warmed safely and the thus embodied apparatus is mechanically simple to realize.

What is claimed is:

1. An apparatus for warming blood or other physiological fluids, comprising a conduit through which the fluid can be conducted, and heating means for warming the fluid flowing through the conduit, wherein the heating means comprise at least one source of infrared radiation, and in that the conduit is at least partly transparent to the radiation emitted by the infrared source during operation, and wherein the conduit at its discharge is provided with a temperature sensor that is coupled to, and feeds a control member, wherein the infrared source is adjustable and connected to the control member.

2. An apparatus according to claim 1, wherein the conduit defines a space wherein the infrared source is placed, and in that the conduit, at least at the side facing the infrared source, is transparent to infrared radiation.

3. An apparatus according to claim 1, wherein the conduit extends substantially in a plane, in that the infrared source is situated next to the conduit, and in that the conduit, at least at the side of the source, is transparent.

4. An apparatus according to claim 1, wherein the conduit extends substantially in the form of a spiral and that its side facing away from the infrared source is provided with an infrared radiation-reflective coating or film.

5. An apparatus according to claim 1, wherein the apparatus is equipped with a flowmeter connected with the control member.

6. An apparatus according to claim 5, wherein the flowmeter comprises a heat source and at least one temperature sensor.

7. An apparatus according to claim 6, characterized in that the flowmeter comprises two temperature sensors and a heat source positioned between said temperature sensors or near the temperature sensor positioned at the discharge side.
